(19) United States
(12) Patent Application Publication
(10) Pub. No.: US 2014/0324063 A1
(43) Pub. Date: Oct. 30, 2014

(54) INTRAOCULAR LENS CARTRIDGE

(71) Applicants: Orhan Haydaroglu, Sehitkamil - Gaziantep (TR); Mustafa Nazlier, Sehitkamil - Gaziantep (TR)

(72) Inventors: Orhan Haydaroglu, Sehitkamil - Gaziantep (TR); Mustafa Nazlier, Sehitkamil - Gaziantep (TR)

(21) Appl. No.: 14/363,047

(22) PCT Filed: Oct. 31, 2012
(86) PCT No.: PCT/TR2012/000182

§ 371 Date: Jun. 5, 2014

(30) Foreign Application Priority Data
Dec. 30, 2011 (TR) 2011/13348

(51) Int. Cl. A61F 2/16

(52) U.S. Cl. CPC A61F 2/1662; A61F 2/16
USPC 606/107

(57) ABSTRACT

Intraocular lens cartridge comprising a conic head portion (1) narrowing gradually after a rectangular prism part; upper channel (1.2) on the inner upper wall of the conic head portion extending until the middle portion of the inner upper wall in order for the lens to move forward properly and continue its folding, lower channel (1.3) on the inner lower wall; upper and lower inverse “L” shaped claws (1.4, 1.5) on the outer surface of the conic head portion; the “L” shaped lower claw having a longer arm; a cartridge body (2) suitable for intertwining, having a larger periphery as compared to the periphery of the head portion of the conic head portion, lock housings (2.2) on the upper and lower parts of the cartridge (bayonet connection between head portion and cartridge body); supporting protrusions (2.1) both on the upper and lower parts for supporting the fingers of the operator.

### TECHNICAL FIELD

The present invention relates to an intraocular lens cartridge used in intraocular lens implantation during a cataract surgery and improvement achieved in the injector structure.

### STATE OF THE ART

Cataract is defined to be a loss of transparency of the crystalline lens. Once the crystalline lens looses its transparency, it becomes smudgy, frosty or like a clouded glass and eyesight complaints emerge. Objects are seen shapeless, cloudy or poor.

The intraocular crystalline lens consists of proteins and water by nature. Characteristic sequence of the proteins renders transparency and light transmissibility to the lens.

Little protein molecules begin to conglomerate as time goes by and eventually the lens loses its transparency. When the opacity dominates over whole lens to the extent that the light cannot be transmitted anymore, mature cataract becomes apparent.

Cataract, neither is the treatment of which possible by medication nor is ceased by using glasses, might be congenital but most frequently appears as a result of ageing. Only remedy to cataract is the surgery. During the development period of the disease, both near and far visual space weakens to an uncomfortable extent.

Surgery is based on a system comprising removal of the opaque crystalline lens and replacement with a new lens or IOL (Intra Ocular Lens).

Present IOL's are made of silicon, soft acrylics and hydrogels which are capable of folding or rolling and being inserted through a smaller incision. Various methods are used during implantation of these lenses into the eyeball in order for them to roll and fold. A common method incorporates an injector cartridge which folds the lenses and through. which a lens can be pushed into the eyeball via a soft-head piston. In the prior art, U.S. Pat. No. 4,681,102 patent document describes a divided cartridge with a longitudinal juncture.

As for EP1980219 (TR2009/08916) in the prior art, these documents mention a cartridge of a lens application system and the method of production of the same which provides a flat cartridge with thin walls and a distal head. By this means, the transition region between the conic folding portion and the head comprises supplementary junction pieces to assist preventing the cartridge from fracturing.

As for US2008/0147081 in the prior art, an intraocular lens injector apparatus was described. An IOL injector kit comprising a first body portion having a first lumen disposed there through, a proximal end, a distal end configured to deposit an IOL into an eye, a second. body portion. having a second lumen disposed there through, a proximal end and distal end, a container comprising a wall defining a container lumen, a first open end configured to receive the proximal end of the first body portion into the container lumen, and a second open end configured to receive the distal end of the second body portion into the container lumen, and an IOL disposed inside said container.

As mentioned hereinabove, certain inconveniences occur in cataract surgeries in the course of applications of the cartridge structures employed. The most important one among these occurs when the lens to be implanted into the eyeball folds and rolls undesirably and is not removed from the cartridge properly when pushed into the cartridge. In each application, employed lenses leave the cartridge in an undesired way, bending and folding with a different angle inside the channel in the cartridge and thus necessitate re-arrangement of the lens during the operation. This in turn decreases the practicality and implies manual intervention, thus extends the duration of the operation.

Present intraocular lens cartridges do not involve a structure intended in support of the operator for an easier handling of the injector and a more sensitive operation.

Another problem is that, during the movement of the injector in presently used intraocular lens cartridges, these two pieces separates uncontrollably, even in the course of the operation.

All inconveniencies of the state of art lens cartridge structures used for intraocular lens implantations mentioned hereinabove are overcome by the improvements of the intraocular lens cartridge according to present invention.

### OBJECT OF THE INVENTION

Aim of the present invention is to enable the lenses to move forward at the same position and to leave the cartridge properly every time, in each operation during the intraocular lens implantation in cataract surgeries.

Another object of the invention is to enable proper implantation of the lens into the eyeball during the operation.

Still another object of the invention is to enable the body and injector structures of the cartridge to interlock.

Yet another object of the invention is to enable ease in handling part of the cartridge and provide ease of use during implantation.

### DESCRIPTION OF THE FIGURES

FIG. 1 is the cross sectional view of the intraocular lens cartridge.

FIG. 2 is the cross view of the body of the intraocular lens cartridge.

FIG. 3 is the lateral sectional view of the conic head portion intraocular lens cartridge.

FIG. 4 is the cross view of the pushing part of the intraocular lens cartridge.

All parts of the intraocular lens cartridge described hereinabove according to the invention are numerated and the names of the parts are given in detail as follows:

1. Conic head portion
1.1. Lens path
1.2. Upper channel
1.3. Lower channel
1.4. Upper claw
1.5. Lower claw
1.6. Body head

2. Cartridge body
2.1. Supporting protrusions
2.2. Lock housing
2.3. Cartridge body head

3. Pushing part

4. Rectangular prism shaped part

5. Narrowing point

6. Head section
A: Head direction

### DESCRIPTION OF THE INVENTION

Present invention relates to an injector employed in the intraocular lens implantation in cataract surgeries, consisting of three separate parts, i.e. a cartridge body (2), a conic head portion (1) and a pushing part (3) used for enabling the lens to leave the head portion properly by applying a driving force.

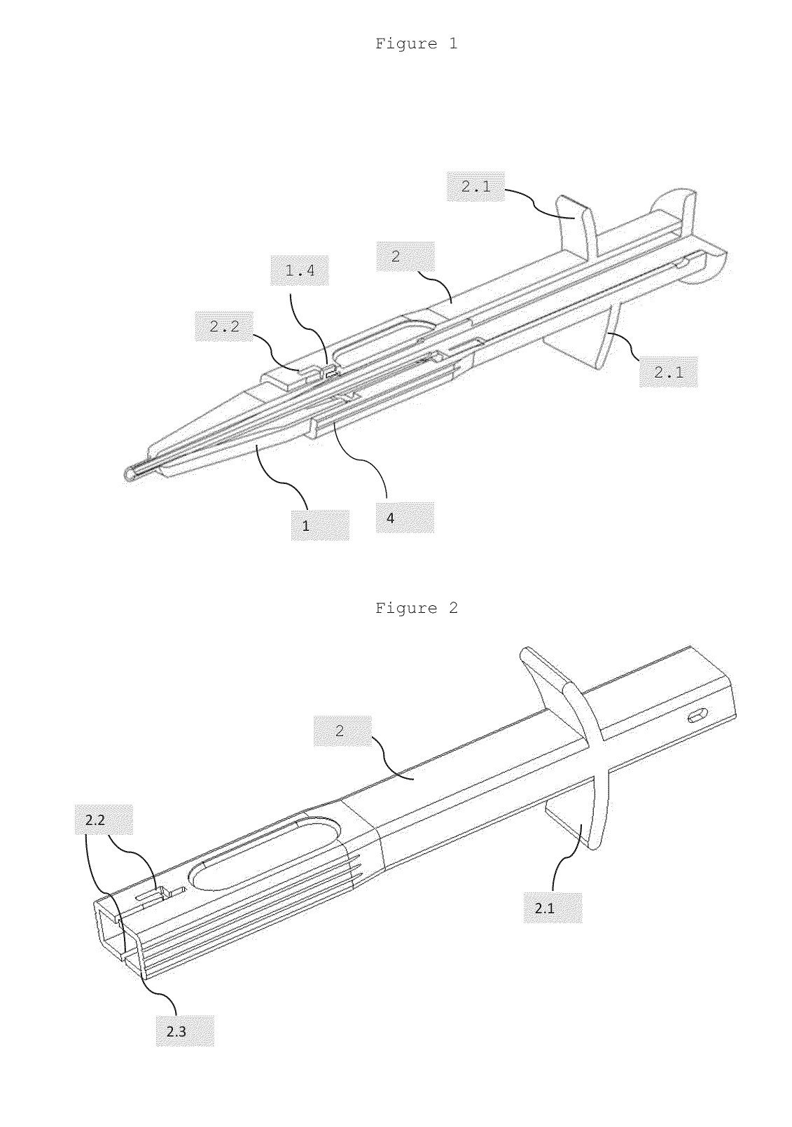

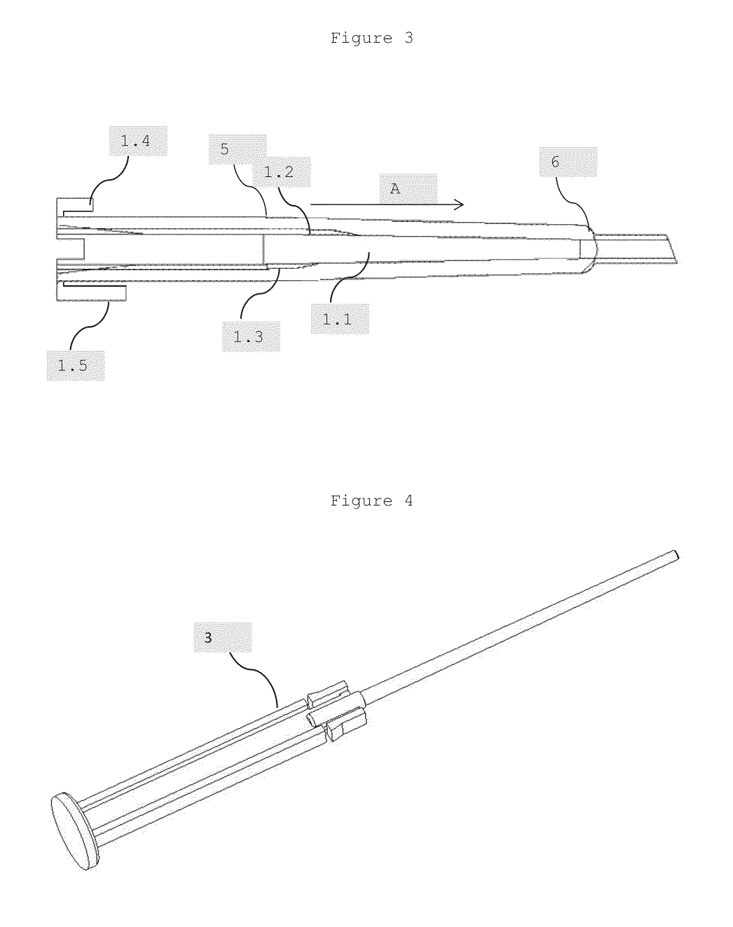

intraocular lens cartridge according to present invention comprises a conic head portion (1) having a hollow portion inside, as seen clearly from FIG. 1, a lens path (1.1) in which the lens is pushed forward in order to be inserted into the eyeball, upper channel (1.2) on the inner upper wall oil the conic head portion (1) and lower channel (1.3) on the inner lower wall extending until the middle portion of the conic head portion (1) in order for the lens to move forward properly in the lens path (1.1) and continue its path folding (FIG. 3), an “L” shaped upper claw (1.4) on the upper outer surface of the conic head portion (1) seen in FIG. 3, an “L” shaped lower claw (1.5) at the opposite position having a longer arm as compared to the upper claw (1.4) on the lower outer surface of the conic head portion (1), lock housings (2.2) placed opposite position (seen clearly from FIG. 2) on the cartridge body head (2.3). The supporting protrusions (2.1) are placed both. on the upper and lower parts of the cartridge body (2), one for each, intended for supporting the fingers of the operator for easily holding the cartridge body (2).

As seen in FIG. 1, the invention comprises a conic portion (1) which narrows gradually after a rectangular prism shaped part (4) and in FIG. 3, it is seen that the upper channel (1.2) on the inner upper wall of the conic head portion (1) ending after a distance from conic narrowing point (5) and extending until the middle portion from the he direction (A) of the conic head portion (1).

Cartridge body (2), suitable for intertwining, has a larger periphery as compared to the periphery as compared of the head section (6) of the conic portion (1).

The cartridge body head (2.3) has a larger periphery as compared. to the periphery of the body head (1.6). So it is possible to connect cartridge body (2) and conic head portion (1) by intertwining. With the intertwining, the cartridge body (2) and the conic head portion (1) locked by attaching the upper claw (1.4) and lower claw (1.5) to lock housing (2.2) (bayonet connection).

Intraocular lens cartridge according to the present invention comprises upper and lower channels (1.2 and 1.3), on the upper and lower portions at the inner wall, ending at a distance from the point at which narrowing begins and which extends from the body head (1.6) to the middle portion of the cartridge, intended for narrowing the lens path (1.1) and enabling the lens to move forward by rendering it a convex shape. Upper channel (1.2) ends at a further distance than does the lower channel (1.3) and thus has a longer structure. As for the lower channel (1.3), since it ends behind, the degree of narrowing between two channels leads the lens to be directed in the horizontal plane after leaving the lower channel (1.3) with a flat slope after a parabolic shape and in this way it continues to move forward on the x-axis evenly and does not deform this flat structure.

Upon insertion of the cartridge body (2) into the body head (1.6), as a result of affixing the upper claw (1.4) and the lower claw (1.5) to the lock housings (2.2) found on the upper and lower portions of the body and as a result of the downward movement thereof, these two portions are interlocked and fixed.

1. Intraocular lens cartridge comprises a conic head portion (1) having a hollow portion inside, lens path (1.1) in which the lens is pushed forward in order to be inserted into the eyeball, a cartridge body (2), a pushing part (3) characterized in that it comprises,
a conic head portion (1) narrowing gradually after a rectangular prism part,
upper channel (1.2) on the inner upper wail of the conic head portion (1) and a lower channel (1.3) on the inner lower wall of the conic head portion (1) extending until the middle portion of the inner. upper wall,
the upper channel (1.2) ends at a distance than does the lower channel (1.3) on the inner upper wall of the conic head portion (1).

2. A conic head portion according to claim 1 characterized in that, having “L” shaped upper and lower claws (1.4, 1.5) on the outer surface.

3. a cartridge body according to claim 1 characterized in that, having a larger periphery as compared to the periphery of the body head (1.6).

4. a cartridge body (2) according to claim 1 characterized in that, having lock housings (2.2) on the cartridge body (2).

5. a cartridge body (2) according to claim 1 characterized in that, having supporting protrusions (2.1) both located on the upper and lower side of the cartridge body (2)
